(19) United States
(12) Patent Application Publication
(10) Pub. No.: US 2006/0194752 A1
(43) Pub. Date: Aug. 31, 2006

(54) Treatment for renal disease

(75) Inventors: Stephen I. Alexander, Sydney (AU); David C. H. Harris, Sydney (AU); Yiping Wang, Sydney (AU); Huiling Wu, Sydney (AU); Guoping Zheng, Sydney (AU)

Correspondence Address: TOWNSEND AND TOWNSEND AND CREW, LLP, TWO EMBARCADERO CENTER, EIGHTH FLOOR, SAN FRANCISCO, CA, 94111-3834, US

(73) Assignee: The Children's Hospital at Westmead Sydney West Area Health Service, Sydney (AU)

(21) Appl. No.: 11/147,500

(22) Filed: Jun. 7, 2005

Related U.S. Application Data
(60) Provisional application No. 60/577,782, filed on Jun. 7, 2004.

(51) Int. Cl. A61K 48/00

(52) U.S. Cl. 514/44

(57) ABSTRACT

Methods for treating or preventing renal disease or inducing passive immunity against renal disease by administering polynucleotides encoding MCP-1 or both MCP-1 and RANTES are provided. Compositions containing polynucleotides encoding MCP-1 or both MCP-1 and RANTES are also provided.

### CROSS-REFERENCES TO RELATED APPLICATIONS

This Application claims the benefit of U.S. Provisional Application No. 60/577,782, filed Jun. 7, 2004, which is incorporated herein by reference in its entirety for all purposes.

### TECHNICAL FIELD

The present invention relates to nucleic acid-based vaccines for the treatment or prevention of renal disease. The invention also relates to methods of treating or preventing renal disease using nucleic acid-based vaccines and to methods of inducing protective immunity against renal disease using such vaccines.

### BACKGROUND OF THE INVENTION

Renal diseases are a significant health issue across the world. In the United States alone it is estimated that approximately 20 million people suffer from chronic renal disease, most commonly diabetic renal disease and hypertensive renal disease. Many renal diseases share a common pathology of tubulointerstitial nephropathy or glomerulonephropathy and a common symptom of proteinuria. Such diseases account for a high percentage of all renal diseases and frequently develop into End-Stage Renal Disease (ESRD). Due to the lack of effective therapies, patients with ESRD typically require dialysis or kidney transplantation.

There is a clear need not only for effective therapies for ESRD but also for treatments of earlier stage renal diseases to prevent the development of ESRD.

Chemokines are pro-inflammatory glycoproteins that have the ability to attract and activate leukocytes. There is growing evidence that chemokines play a central role in inflammation and various diseases, including renal diseases. Increased expression of chemokines has been found in both human renal diseases and in animal models of acute glomerular or tubulointerstitial diseases (Sergerer, 2003; Anders et al, 2003). The selective role of chemokines in the trafficking of macrophages and T cells to sites of inflammation may be crucial in the evolution of renal injury as chemokine expression correlates with the local infiltration of effector cells and renal damage. In particular, in vivo studies in animals and humans suggest a pivotal role for the C-C chemokines monocycle chemoattractant protein 1 (MCP-1) and RANTES in producing renal inflammation (see, for example, Wang et al, 1997; Rangan et al, 2000). Studies in different immunologic models of renal disease suggest pathogenic roles for MCP-1 and RANTES in the recruitment of leukocytes into the interstitium and the resulting tissue damage in chronic proteinuric renal disease.

Accordingly, methods for the blockage or inhibition of chemokine activity or chemokine-dependent pathways may offer therapeutic potential for the treatment of renal diseases. Blocking chemokine activity using neutralizing antibodies has been demonstrated to offer some protection in several models of renal injury. For example, treatment with MCP-1 antibodies reduced proteinuria and monocyte infiltration in rat nephrotoxic serum nephritis (Lloyd et al, 1997). However a major limitation in the treatment of chronic diseases with neutralizing antibodies is their immunogenicity, that is the development of host antibodies to the therapeutic antibodies. Indeed the therapeutic application of anti-chemokine antibodies has proved to be ineffective or of limited effectiveness in renal disease treatment (Fujinaka et al, 1997; Wu et al, 1997). The redundancy of chemokines and chemokine receptors and the generation of host antibodies to the therapeutic antibodies, particularly following multiple administrations, may be significant factors limiting the effectiveness of antibody therapy for renal disease.

There is a clear need for improved compositions and methods for inhibiting chemokine expression and treating renal diseases.

The present invention is predicated on the inventors' findings that vaccination using naked DNA encoding MCP-1 and/or RANTES ameliorates the progression of renal disease in the rat adriamycin nephropathy model of chronic proteinuric renal disease.

### BRIEF SUMMARY OF THE INVENTION

According to one aspect of the present invention there is provided a method for the treatment or prevention of renal disease in a subject, the method comprising administering to the subject an effective amount of a polynucleotide encoding MCP-1 operably linked to a promoter.

The polynucleotide encoding MCP-1 may have the nucleotide sequence set forth in SEQ ID NO: 1.

The polynucleotide may encode a hybrid MCP-1 polypeptide. In the hybrid polypeptide a surface loop region of MCP-1 may be replaced with a corresponding region of a second protein. In the hybrid polypeptide a surface loop region of MCP-1 may be replaced with the P30 tetanus toxoid T helper epitope.

In one embodiment the polynucleotide encoding hybrid MCP-1 has the nucleotide sequence set forth in SEQ ID NO:5.

The hybrid MCP-1 polypeptide may have the amino acid sequence set forth in SEQ ID NO:6.

The method may further comprise administering to the subject an effective amount of a polynucleotide encoding RANTES operably linked to a promoter. The polynucleotide encoding RANTES may have the nucleotide sequence set forth in SEQ ID NO:3.

The renal disease may be a chronic proteinuric renal disease. The renal disease may be selected from the group consisting of: focal glomerulosclerosis, glomerulonephritis, diabetic renal disease, hypertensive renal disease, renal failure, end-stage renal disease, or a related condition.

The polynucleotide encoding MCP-1 and the polynucleotide encoding RANTES may be located in a single nucleic acid construct.

Administration of the polynucleotide(s) may induce an immune response in the subject.

According to another aspect of the present invention there is provided a method for the treatment or prevention of renal disease in a subject, the method comprising administering to the subject an effective amount of a polynucleotide encoding MCP-1 operably linked to a promoter and a polynucleotide encoding RANTES operably linked to a promoter.

The polynucleotide encoding MCP-1 may have the nucleotide sequence set forth in SEQ ID NO:1. The polynucleotide encoding RANTES may have the nucleotide sequence set forth in SEQ ID NO:3.

MCP-1 may have the amino acid sequence as set forth in SEQ ID NO:2. RANTES may have the amino acid sequence as set forth in SEQ ID NO:4.

The MCP-1 encoding polynucleotide may encode a hybrid MCP-1 polypeptide. In the hybrid polypeptide a surface loop region of MCP-1 may be replaced with a corresponding region of a second protein. In the hybrid polypeptide a surface loop region of MCP-1 may be replaced with the P30 tetanus toxoid T helper epitope. The polynucleotide encoding the hybrid MCP-1 may have the nucleotide sequence set forth in SEQ ID NO:5. The hybrid MCP-1 polypeptide may have the amino acid sequence set forth in SEQ ID NO:6.

The subject may be human.

The renal disease may be a chronic proteinuric renal disease. The renal disease may be selected from the group consisting of: focal glomerulosclerosis, glomerulonephritis, diabetic renal disease, hypertensive renal disease, renal failure, end-stage renal disease, or a related condition.

The polynucleotide encoding MCP-1 and the polynucleotide encoding RANTES may be located in a single nucleic acid construct. The nucleic acid construct may be a DNA construct. The DNA construct may be a plasmid.

In an embodiment, the administration of the polynucleotides induces an immune response in the subject.

According to a further aspect of the present invention there is provided a method for inducing protective immunity against renal disease in a subject, the method comprising administering to the subject an effective amount of a polynucleotide encoding MCP-1 operably linked to a promoter.

The polynucleotide may encode a wild-type MCP-1 polypeptide or a modified MCP-1 polypeptide. The polynucleotide encoding MCP-1 may have the nucleotide sequence set forth in SEQ ID NO:1 or SEQ ID NO:5.

According to a further aspect of the present invention there is provided a method for inducing protective immunity against renal disease in a subject, the method comprising administering to the subject an effective amount of a polynucleotide encoding MCP-1 operably linked to a promoter and a polynucleotide encoding RANTES operably linked to a promoter.

According to a further aspect of the present invention there is provided an immunological composition comprising a polynucleotide encoding MCP-1, wherein administration of the composition to a subject induces an immune response in the subject.

The polynucleotide may encode a wild-type MCP-1 polypeptide or a modified MCP-1 polypeptide. The polynucleotide encoding MCP-1 may have the nucleotide sequence set forth in SEQ ID NO:1 or SEQ ID NO:5.

According to a further aspect of the present invention there is provided an immunological composition comprising a polynucleotide encoding MCP-1 and a polynucleotide encoding RANTES, wherein administration of the composition to a subject induces an immune response in the subject.

According to a further aspect of the present invention there is provided a composition for the treatment or prevention of renal disease comprising a polynucleotide encoding MCP-1 operably linked to a promoter.

The polynucleotide may encode a wild-type MCP-1 polypeptide or a modified MCP-1 polypeptide. The polynucleotide encoding MCP-1 may have the nucleotide sequence set forth in SEQ ID NO:1 or SEQ ID NO:5.

According to a further aspect of the present invention there is provided a composition for the treatment or prevention of renal disease comprising a polynucleotide encoding MCP-1 and a polynucleotide encoding RANTES, the polynucleotides being operably linked to a promoter.

According to the above aspects, the compositions of the invention may optionally further comprise one or more pharmaceutically acceptable carriers, diluents or adjuvants.

### Definitions

As used herein, the term “protective immunity” refers to the ability of a molecule or composition administered to a subject to elicit an appropriate immune response in the subject and thereby provide protection to the subject from the development or progression of renal disease.

The term “polynucleotide” as used herein refers to a single- or double-stranded polymer of deoxyribonucleotide, ribonucleotide bases or known analogues of natural nucleotides, or mixtures thereof. The term “polypeptide” means a polymer made up of amino acids linked together by peptide bonds.

As used herein, the term “nucleic acid” refers to a deoxyribonucleotide or ribonucleotide polymer in either single- or double-stranded form, and unless otherwise limited, encompasses known analogues of natural nucleotides that hybridize to nucleic acids in a manner similar to naturally occurring nucleotides.

As used herein the term “treatment” refers to any and all uses which remedy a disease state or symptoms, prevent the establishment of disease, or otherwise prevent, hinder, retard, or reverse the progression of disease or other undesirable symptoms in any way whatsoever.

As used herein the term “effective amount” includes within its meaning a non-toxic but sufficient amount of an agent or compound to provide the desired therapeutic effect. The exact amount required will vary from subject to subject depending on factors such as the species being treated, the age and general condition of the subject, the severity of the condition being treated, the particular agent being administered and the mode of administration and so forth. Thus, it is not possible to specify an exact “effective amount”. However, for any given case, an appropriate “effective amount” may be determined by one of ordinary skill in the art using only routine experimentation.

In the context of this specification, the term “comprising” means “including principally, but not necessarily solely”. Furthermore, variations of the word “comprising”, such as “comprise” and “comprises”, have correspondingly varied meanings.

### BRIEF DESCRIPTION OF THE DRAWINGS

Embodiments of the present invention will now be described, by way of example only, with reference to the accompanying drawings.

FIG. 1 MCP-1 and RANTES mRNA expression in AN kidney measured by semiquantitative RT-PCR. Results are expressed as the mean ratio of chemokine gene densitometry score to 18s rRNA densitometry score±SD. N=5 per group.

FIG. 2. Proteinuria week 1 to week 4 post adriamycin administration following vaccination with a DNA vaccine encoding MCP-1 and RANTES. DV: DNA vaccination against MCP-1 and RANTES in AN (◯); DV control: DNA vaccination with empty pTarget vector as a control for vaccination in AN (▪); AN: injection with adriamycin alone (●); PBS: injection with PBS only in normals as a control for adriamycin (□). Mean±SD. (*P<0.05, ** P<0.01).

FIG. 3. Creatinine clearance (ml/min) following vaccination with a DNA vaccine encoding MCP-1 and RANTES. DV, DV control (ctrl) and AN as for FIG. 2. Mean±SD. (* p<0.05).

FIG. 4. Kidney sections at week 4 post adriamycin administration observed under the light microscope following vaccination with a DNA vaccine encoding MCP-1 and RANTES. Sections stained by periodic acid Schiff in DV group (A), DV control (B), AN (C) and PBS (D). (Magnification 200×).

FIG. 5. Renal interstitial infiltrates at week 4 post adriamycin administration following vaccination with a DNA vaccine encoding MCP-1 and RANTES. A. CD68+ Macrophages, CD8+ and CD4+ T cells, and CD25+ infiltrates in renal interstitium in the DV, DV control, AN and PBS groups (*p<0.0001). Results are expressed as mean±SD of cells per 400× field. B. Representative immunoperoxidase-stained kidney sections from the AN and DV groups. (Magnification 200×).

FIG. 6. Antibody production (MCP-1 antibody and RANTES antibody) measured by ELISA in sera of AN rats following vaccination with a DNA vaccine encoding MCP-1 and RANTES. (*P<0.005).

FIG. 7. 3D structure prediction of modified MCP-1 protein by SWISS-MODEL. A. Wild-type MCP-1: peptide in yellow (bounded by white arrows) is targeted for modification. B. Modified MCP-1: peptide in yellow (bounded by white arrows) is the replacing P30 sequence.

FIG. 8. Expression of modified MCP-1 protein in vitro. HeLa cells were transiently transfected with plasmid constructs of modified and unmodified MCP-1 DNA vaccines as well as pTarget vector control. Lysates of transfected HeLa cells were probed with rabbit anti-rat MCP-1 polyclonal Ab. Lane 1, HeLa cells transfected with modified MCP-1 DNA vaccine; lane 2, HeLa cells transfected with unmodified MCP-1 DNA vaccine; lane 3, HeLa cells transfected with pTarget vector control; lane 4, HeLa cell lysate control.

FIG. 9. Protection against renal structural injury in AN rats by vaccination with modified MCP-1 DNA vaccine. Kidney tissues harvested 4 weeks after ADR administration were analysed under PAS stain. Representative histology of: A. normal control; B. rats vaccinated with vector control; C. rats vaccinated with modified MCP-1 DNA; D. rats vaccinated with unmodified MCP-1 DNA.

FIG. 10. Creatinine clearance (ml/min) week 1 to week 4 post adriamycin administration following vaccination with a DNA vaccine encoding wild-type MCP-1 (▪), modified MCP-1 (▴), or empty vector control (♦). Mean±SD. (*p<0.05).

FIG. 11. Self-specific Ab titer to MCP-1 determined by ELISA using serial dilution of sera of DNA vaccinated rats. The Ab titer in sera of rats vaccinated with modified MCP-1 DNA was significantly higher at week 2 compared to that of rats vaccinated with unmodified MCP-1 at week 2 (†P<0.05), and to each other groups at week 0 and 2 (**P<0.001). The Ab titer in sera of modified MCP-1 vaccinated rats without ADR was not different from that of normal control at both week 0 and 2. Results are means±SE.

FIG. 12. Self-specific Ab to MCP-1 determined by ELISA using one dilution of sera of DNA vaccinated rats (1:100). Positive binding (%) was measured by OD at 405 nm corrected for the OD of known positive serum. High levels of anti-MCP-1 Ab were produced by rats vaccinated with modified or unmodified MCP-1 DNA at 2 and 4 weeks after ADR. The results are means±SE. * P<0.05 vs vector control, modified MCP-1 vaccine alone and AN alone.

FIG. 13. Self-specific Ab to MIP-1α determined by ELISA using one dilution of sera of DNA vaccinated rats (1:100). Positive binding (%) was measured by OD at 405 nm corrected of the OD of known positive serum. High levels of anti-MIP-1α Ab were produced by rats vaccinated with modified or unmodified MCP-1 DNA at 2 and 4 weeks after ADR. The results are means±SE of three different sera. * P<0.05 vs vector control, modified MCP-1 vaccine alone and AN alone.

FIG. 14. IFN-γ producing T cells (from draining lymph nodes, DLN) as determined by ELISpot in response to rMCP-1 stimulation in vitro. Results are the number of spot-forming cells (SFC) per 10⁶ DLN cells, expressed as mean±SE of three different samples. * P<0.05 compared with unmodified MCP-1 and vector vaccination and normal control.

FIG. 15. Number of interstitial inflammatory cells in kidney, determined by immunohistochemistry. Results are the average number of cells counted from 20 of ×400 fields per animal. Values are ±SE. ** P<0.001 vs other 3 groups (unmodified MCP-1, vector control and AN alone).

FIG. 16. Blockade of monocyte chemotaxis by sera of DNA vaccinated rats, as assessed by total number of cells migrating to lower chamber through 8 μM membrane in response to rMCP-1. Values are means±SE of three different serum samples. ** P<0.01 vs unmodified MCP-1 and vector control.

### DETAILED DESCRIPTION OF THE INVENTION

Adriamycin nephropathy (AN) is an animal model of human renal disease. AN is induced in mice and rats by a single intravenous injection of adriamycin, which is directly toxic to the kidney, leading to chronic proteinuric renal disease similar to human focal segmental glomerulosclerosis (Rangan et al, 1999). As disclosed herein, the present inventors have shown that using a novel therapeutic approach, that of vaccination using DNA encoding MCP-1 or using DNA encoding both MCP-1 and RANTES significantly reduces proteinuria, interstitial infiltrates (specifically T cells and macrophages) and protects against renal injury in AN. Further, vaccination using DNA encoding MCP-1 and RANTES, produces significantly higher levels of specific antibodies to MCP-1 and RANTES than in the absence of vaccination. Vaccination using DNA encoding a modified MCP-1 protein alone also provided enhanced protection over that achieved using DNA encoding wild-type MCP-1. Enhanced production of autoantibody against native MCP-1 protein, as well as the number of IFN-γ producing T cells from draining lymph nodes was also observed.

Accordingly, one aspect of the present invention relates to a method for the treatment or prevention of renal disease in a subject, the method comprising administering to the subject an effective amount of a polynucleotide encoding MCP-1 operably linked to a promoter.

Another aspect of the present invention relates to a method for the treatment or prevention of renal disease in a subject, the method comprising administering to the subject an effective amount of a polynucleotide encoding MCP-1 operably linked to a promoter and a polynucleotide encoding RANTES operably linked to a promoter.

The invention further relates to a method for inducing protective immunity against renal disease using the polynucleotides described herein and to compositions for inducing an immune response or for treating or preventing renal disease, the compositions comprising one or more of the polynucleotides of these aspects.

In particular embodiments, the polynucleotides are administered to subjects in a vector. The vector may be a plasmid vector, a viral vector, or any other suitable vehicle adapted for the insertion of foreign sequences, their introduction into eukaryotic cells and the expression of the introduced sequences. Typically the vector is a eukaryotic expression vector and may include expression control and processing sequences such as a promoter, an enhancer, ribosome binding sites, polyadenylation signals and transcription termination sequences.

In embodiments in which both the MCP-1 and RANTES-encoding polynucleotides are administered, the polynucleotides may be located on separate nucleic acid constructs or on the same construct. In embodiments in which the polynucleotides are located on the same construct, they may be operably linked to the same of different promoters.

A nucleic acid construct in accordance with an embodiment of the present invention may comprise a vaccine, in particular a DNA vaccine. Accordingly, the present invention also relates to methods of DNA vaccination of subjects for the treatment or prevention of renal disease or to induce protective immunity against renal disease. The DNA vaccine may comprise naked DNA or may be in the form of a composition, together with one or more pharmaceutically acceptable carriers.

MCP-1 and RANTES Sequences

Those skilled in the art will appreciate that the precise sequences of the MCP-1-encoding and RANTES-encoding polynucleotides used according to the methods and compositions of the present invention may vary depending on a number of factors, for example the species of animal to be treated such that the sequences of the MCP-1 and RANTES polynucleotides are selected so as to be derived from the species to be treated. For example, in the treatment of human renal diseases polynucleotides encoding the human MCP-1 and RANTES chemokines may be used.

In a particular embodiment, the nucleotide sequence of the polynucleotide encoding MCP-1 is as set forth in SEQ ID NO:1 or a fragment or variant thereof, or displays sufficient sequence identity thereto to hybridise to the sequence of SEQ ID NO:1. In alternative embodiments, the nucleotide sequence of the polynucleotide may share at least 30%, 40%, 50%, 60%, 70%, 80%, 85%, 90%, 96%, 97%, 98% or 99% identity with the sequence set forth in SEQ ID NO:1.

In a particular embodiment, the nucleotide sequence of the polynucleotide encoding RANTES is as set forth in SEQ ID NO:3 or a fragment or variant thereof, or displays sufficient sequence identity thereto to hybridise to the sequence of SEQ ID NO:3. In alternative embodiments, the nucleotide sequence of the polynucleotide may share at least 30%, 40%, 50%, 60%, 70%, 80%, 85%, 90%, 96%, 97%, 98% or 99% identity with the sequence set forth in SEQ ID NO:3.

The polynucleotide encoding MCP-1 may encode a polypeptide having the amino acid sequence as set forth in SEQ ID NO:2. The polynucleotide encoding RANTES may encode a polypeptide having the amino acid sequence as set forth in SEQ ID NO:4. Within the scope of the term “polypeptide” as used herein are fragments and variants thereof.

The term “fragment” refers to a nucleic acid or polypeptide sequence that encodes a constituent or is a constituent of the full-length MCP-1 or RANTES chemokines. In terms of the polypeptide the fragment may possesses qualitative biological activity in common with the full-length chemokine, or may be an immunogenic portion thereof, capable of inducing protective immunity against renal disease in a subject.

The term “variant” as used herein refers to substantially similar sequences. Generally, nucleic acid sequence variants encode polypeptides which possess qualitative biological activity in common. Generally, polypeptide sequence variants also possess qualitative biological activity in common. Further, these polypeptide sequence variants may share at least 25%, 30%, 35%, 40%, 45%, 50%, 55%, 60%, 65%, 70%, 75%, 80%, 85%, 90%, 95%, 96%, 97%, 98% or 99% sequence identity.

Those skilled in the art will also readily appreciate that various modifications may be made to the sequences of the polynucleotides encoding MCP-1 and RANTES such that modified variants of the MCP-1 and RANTES polypeptides are generated in which one or more portions of the encoded polypeptide is replaced by one or more portions of another polypeptide. The portion(s) replaced may correspond to a particular structural or functional domain of the MCP-1 or RANTES polypeptide. Such modifications are also included within the scope of the term “variant”. For example, modifications may be made so as to enhance the immuno-protective properties of the MCP-1 or RANTES polypeptide or to otherwise increase the effectiveness of the polypeptide to treat or prevent renal disease.

In one such example as disclosed herein, the polynucleotide encoding MCP-1 may be modified by PCR or other suitable technique to replace a surface loop region of MCP-1 with a region of an alternative protein, such as the P30 tetanus toxoid T helper epitope thereby producing a hybrid protein (referred to herein as “modified MCP-1”). T cell help has been considered crucial in breaking B cell tolerance to permit development of auto-antibodies against self-proteins. The foreign T helper epitope has been reported to break B cell tolerance to the highly conserved self-protein ubiquitin when expressed as a hybrid protein with ubiquitin (Dalum et al., 1996) and P30 from tetanus toxoid was also reported to bypass immunological tolerance in a vaccine against IL-5 (Hertz et al., 2001).

In one particular embodiment of the present invention the polynucleotide sequence encoding the modified MCP-1 may have the nucleotide sequence as set forth in SEQ ID NO:5 or a variant thereof. The modified MCP-1 polypeptide may have the amino acid sequence set forth in SEQ ID NO:6 or a variant thereof.

It will be appreciated by those skilled in the art that the RANTES polynucleotide and polypeptide may be similarly modified.

Further, a variant polypeptide may include analogues, wherein the term “analogue” means a polypeptide which is a derivative of MCP-1 or RANTES, which derivative comprises addition, deletion, substitution of one or more amino acids, such that the polypeptide retains substantially the same function as native MCP-1 or RANTES. The term “conservative amino acid substitution” refers to a substitution or replacement of one amino acid for another amino acid with similar properties within a polypeptide chain (primary sequence of a protein). For example, the substitution of the charged amino acid glutamic acid (Glu) for the similarly charged amino acid aspartic acid (Asp) would be a conservative amino acid substitution.

It will be appreciated that in accordance with aspects and embodiments of the present invention, polynucleotide encoding wild-type or modified MCP-1 may be administered together with polynucleotide encoding the wild-type or modified RANTES.

Vaccines

Compositions comprising polynucleotides encoding MCP-1 and/or RANTES may be administered in the form of a nucleic-acid based vaccine, in particular a DNA vaccine. The DNA vaccine may comprise naked DNA comprising one or more of the polynucleotides as defined herein.

A major limitation in treatment of chronic diseases with neutralizing antibodies is their immunogenicity (that is, the development of host anti-antibodies against therapeutic antibodies). DNA vaccination, inducing immunization with plasmid DNA encoding antigen, represents a novel means of expressing antigen in vivo for the generation of both cellular and humoral immune responses against products of a given construct. Naked DNA vaccines promote a highly efficient protective immunity not only against foreign antigens, such as microbes and tumours but also self antigens, such as TCR V genes. A distinct advantage of DNA vaccines is the induction of cellular or humoral immune response to autologous antigen. In addition, when co-delivered with plasmid-DNA encoding other molecules, there is the possibility of enhancement or modulation of the subsequent response to the DNA encoded antigen.

A typical vaccination regime is to deliver the vaccine in multiple doses, generally one, two or more equal doses.

Vaccination using nucleic acid-based vaccines according to the invention may provide protective immunity against renal disease to the subject being vaccinated. That is, the polypeptide(s) encoded by the nucleic acid vaccine may elicit a protective immune response in the subject, for example by inducing the production of autoantibodies against the encoded polypeptide(s). In this regard, the person skilled in the art will readily appreciate that not only full-length MCP-1 and RANTES polynucleotide sequences may be used in a vaccine, but also fragments or variants thereof, wherein the fragments or variants are capable of encoding immunogenic polypeptides to elicit an immune response and thereby provide protective immunity against renal disease.

The efficiency of nucleic acid uptake into cells can be increased by pre-treatment of cells with one or more enhancing agents capable of enhancing the cellular uptake of nucleic acids. Many suitable agents are known to those skilled in the art. One such agent is bupivacaine, commonly used to enhance the efficiency of transduction of naked DNA. The person skilled in the art will readily appreciate which nucleic acid uptake-enhancing agents may be suitable and the appropriate concentrations of these agents. Further, the nucleic acid(s) may be administered in the presence of one or more delivery vehicles or agents, for example liposomes or a cationic lipid such as lipofectamine. A number of suitable delivery agents or vehicles are known to those skilled in the art.

Compositions and Routes of Administration

Polynucleotides encoding MCP-1 and/or RANTES may be administered in the form of a composition, together with one or more pharmaceutically acceptable carriers. Compositions may be administered either therapeutically or preventively. In a therapeutic application, compositions are administered to a patient already suffering from a renal disease, in an amount sufficient to cure or at least partially arrest the disease and its complications. The composition should provide a quantity of the compound or agent sufficient to effectively treat the patient. Typically in therapeutic applications the treatment would be for the duration of the disease state.

The therapeutically effective dose level for any particular patient will depend upon a variety of factors including: the disorder being treated and the severity of the disorder; activity of the compound or agent employed; the composition employed; the age, body weight, general health, sex and diet of the patient; the time of administration; the route of administration; the rate of sequestration of the agent or compound; the duration of the treatment; drugs used in combination or coincidental with the treatment, together with other related factors well known in medicine.

One skilled in the art would be able, by routine experimentation, to determine an effective, non-toxic amount of agent or compound which would be required to treat applicable diseases.

Generally, an effective dosage is expected to be in the range of about 0.0001 mg to about 1000 mg per kg body weight per 24 hours; typically, about 0.001 mg to about 750 mg per kg body weight per 24 hours; about 0.01 mg to about 500 mg per kg body weight per 24 hours; about 0.1 mg to about 500 mg per kg body weight per 24 hours; about 0.1 mg to about 250 mg per kg body weight per 24 hours; about 1.0 mg to about 250 mg per kg body weight per 24 hours. More typically, an effective dose range is expected to be in the range about 1.0 mg to about 200 mg per kg body weight per 24 hours; about 1.0 mg to about 100 mg per kg body weight per 24 hours; about 1.0 mg to about 50 mg per kg body weight per 24 hours; about 1.0 mg to about 25 mg per kg body weight per 24 hours; about 5.0 mg to about 50 mg per kg body weight per 24 hours; about 5.0 mg to about 20 mg per kg body weight per 24 hours; about 5.0 mg to about 15 mg per kg body weight per 24 hours.

Alternatively, an effective dosage may be up to about 500 mg/m². Generally, an effective dosage is expected to be in the range of about 25 to about 500 mg/m², preferably about 25 to about 350 mg/m², more preferably about 25 to about 300 mg/m², still more preferably about 25 to about 250 mg/m², even more preferably about 50 to about 250 mg/m², and still even more preferably about 75 to about 150 mg/m².

Further, it will be apparent to one of ordinary skill in the art that the optimal quantity and spacing of individual dosages will be determined by the nature and extent of the disease state being treated, the form, route and site of administration, and the nature of the particular individual being treated. Also, such optimum conditions can be determined by conventional techniques.

It will also be apparent to one of ordinary skill in the art that the optimal course of treatment, such as, the number of doses of the composition given per day for a defined number of days, can be ascertained by those skilled in the art using conventional course of treatment determination tests.

In general, suitable compositions may be prepared according to methods which are known to those of ordinary skill in the art and accordingly may include a pharmaceutically acceptable carrier, diluent and/or adjuvant. These compositions can be administered by standard routes. In general, the compositions may be administered by the parenteral (e.g., intravenous, intraspinal, subcutaneous or intramuscular) or oral route. More preferably administration is by the parenteral route, in particular intramuscularly.

The carriers, diluents and adjuvants must be “acceptable” in terms of being compatible with the other ingredients of the composition, and not deleterious to the recipient thereof.

Examples of pharmaceutically acceptable carriers or diluents are demineralised or distilled water; saline solution; vegetable based oils such as peanut oil, safflower oil, olive oil, cottonseed oil, maize oil, sesame oils such as peanut oil, safflower oil, olive oil, cottonseed oil, maize oil, sesame oil, arachis oil or coconut oil; silicone oils, including polysiloxanes, such as methyl polysiloxane, phenyl polysiloxane and methylphenyl polysolpoxane; volatile silicones; mineral oils such as liquid paraffin, soft paraffin or squalane; cellulose derivatives such as methyl cellulose, ethyl cellulose, carboxymethylcellulose, sodium carboxymethylcellulose or hydroxypropylmethylcellulose; lower alkanols, for example ethanol or iso-propanol; lower aralkanols; lower polyalkylene glycols or lower alkylene glycols, for example polyethylene glycol, polypropylene glycol, ethylene glycol, propylene glycol, 1,3-butylene glycol or glycerin; fatty acid esters such as isopropyl palmitate, isopropyl myristate or ethyl oleate; polyvinylpyrolidone; agar; gum tragacanth or gum acacia, and petroleum jelly. Typically, the carrier or carriers will form from 10% to 99.9% by weight of the compositions.

The compositions of the invention may be in a form suitable for administration by injection, in the form of a formulation suitable for oral ingestion (such as capsules, tablets, caplets, elixirs, for example), in the form of an ointment, cream or lotion suitable for topical administration, in a form suitable for delivery as an eye drop, in an aerosol form suitable for administration by inhalation, such as by intranasal inhalation or oral inhalation, in a form suitable for parenteral administration, that is, subcutaneous, intramuscular or intravenous injection.

For administration as an injectable solution or suspension, non-toxic parenterally acceptable diluents or carriers can include, Ringer's solution, isotonic saline, phosphate buffered saline, ethanol and 1,2 propylene glycol.

Some examples of suitable carriers, diluents, excipients and adjuvants for oral use include peanut oil, liquid paraffin, sodium carboxymethylcellulose, methylcellulose, sodium alginate, gum acacia, gum tragacanth, dextrose, sucrose, sorbitol, mannitol, gelatine and lecithin. In addition these oral formulations may contain suitable flavouring and colourings agents. When used in capsule form the capsules may be coated with compounds such as glyceryl monostearate or glyceryl distearate which delay disintegration.

Adjuvants typically include emollients, emulsifiers, thickening agents, preservatives, bactericides and buffering agents.

Solid forms for oral administration may contain binders acceptable in human and veterinary pharmaceutical practice, sweeteners, disintegrating agents, diluents, flavourings, coating agents, preservatives, lubricants and/or time delay agents. Suitable binders include gum acacia, gelatine, corn starch, gum tragacanth, sodium alginate, carboxymethylcellulose or polyethylene glycol. Suitable sweeteners include sucrose, lactose, glucose, aspartame or saccharine. Suitable disintegrating agents include corn starch, methylcellulose, polyvinylpyrrolidone, guar gum, xanthan gum, bentonite, alginic acid or agar. Suitable diluents include lactose, sorbitol, mannitol, dextrose, kaolin, cellulose, calcium carbonate, calcium silicate or dicalcium phosphate. Suitable flavouring agents include peppermint oil, oil of wintergreen, cherry, orange or raspberry flavouring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

Liquid forms for oral administration may contain, in addition to the above agents, a liquid carrier. Suitable liquid carriers include water, oils such as olive oil, peanut oil, sesame oil, sunflower oil, safflower oil, arachis oil, coconut oil, liquid paraffin, ethylene glycol, propylene glycol, polyethylene glycol, ethanol, propanol, isopropanol, glycerol, fatty alcohols, triglycerides or mixtures thereof.

Suspensions for oral administration may further comprise dispersing agents and/or suspending agents. Suitable suspending agents include sodium carboxymethylcellulose, methylcellulose, hydroxypropylmethyl-cellulose, poly-vinyl-pyrrolidone, sodium alginate or acetyl alcohol. Suitable dispersing agents include lecithin, polyoxyethylene esters of fatty acids such as stearic acid, polyoxyethylene sorbitol mono- or di-oleate, -stearate or -laurate, polyoxyethylene sorbitan mono- or di-oleate, -stearate or -laurate and the like.

The emulsions for oral administration may further comprise one or more emulsifying agents. Suitable emulsifying agents include dispersing agents as exemplified above or natural gums such as guar gum, gum acacia or gum tragacanth.

Methods for preparing parenterally administrable compositions are apparent to those skilled in the art, and are described in more detail in, for example, Remington's Pharmaceutical Science, 15th ed., Mack Publishing Company, Easton, Pa., hereby incorporated by reference herein.

The vaccines and other compositions of the present invention may be administered in combination with other therapies for the treatment or prevention of renal disease. For example, a vaccine or composition of the invention may be administered in combination with other agents known to assist in the reduction or prevention of proteinuria or interstitial infiltrates. For such combination therapies, each component of the combination therapy may be administered at the same time, or sequentially in any order, or at different times, so as to provide the desired effect. Alternatively, the components may be formulated together in a single dosage unit as a combination product. When administered separately, it may be preferred for the components to be administered by the same route of administration, although it is not necessary for this to be so. Similarly a vaccine or composition of the invention may be administered in combination with other renal disease treatment regimes such as dialysis.

Renal Diseases

The methods and compositions of the present invention are applicable to the treatment and prevention of various renal diseases and renal injury, both primary and secondary. Secondary renal diseases include those diseases resulting from a pre-existing condition such as diabetes or hypertension. The methods and compositions of the present invention find application particularly in chronic inflammatory and autoimmune diseases characterised by pathological changes of tubulointerstitial nephropathy or glomerulonephropathy and by proteinuria. Diseases include, but are not limited to, focal segmental glomerulosclerosis, glomerulonephritis, diabetic renal disease, hypertensive renal disease, renal failure, End Stage Renal Disease, and related conditions.

The present invention will now be described with reference to specific examples, which should not be construed as in any way limiting the scope of the invention.

### EXAMPLES

Protocols for experiments described herein were approved by the conjoint Animal Ethics Committee of the Children's Medical Research Institute and The Children's Hospital at Westmead, Sydney, Australia.

### Example 1

### High Levels of MCP-1 and RANTES mRNA Expression in AN Kidney

Adriamycin nephropathy (AN), an animal model of human renal disease displays pathologic features including severe nephrotic syndrome, focal glomerular sclerosis and tubular injury with massive mononuclear cell infiltrates composed largely of macrophages and T cells. Previous studies have demonstrated that in AN overt proteinuria appears shortly after adriamycin administration, glomerular vacuolation and focal glomerular sclerosis appear by week 4 and extensive focal and global glomerular sclerosis by week 6. Tubulointerstitial infiltrates are dominated by macrophages at week 2, and later by accumulation of both CD⁺4 and CD⁺8 T cells (Rangan et al, 1999, 2001; Wang et al, 2000).

Inbred male Wistar rats were obtained from the Animal Care Facility, Westmead Hospital, Sydney, Australia. Rats weighing 130-180 g at the age of 5-6 weeks were used in all experiments. AN was induced as described previously (Rangan et al, 2001). Rats received a single intracardiac injection of adriamycin (6.9 mg/kg; David Bull Laboratories, Victoria, Australia). Rats were kept in individual cages and allowed free access to water and regular rat chow ad libitum.

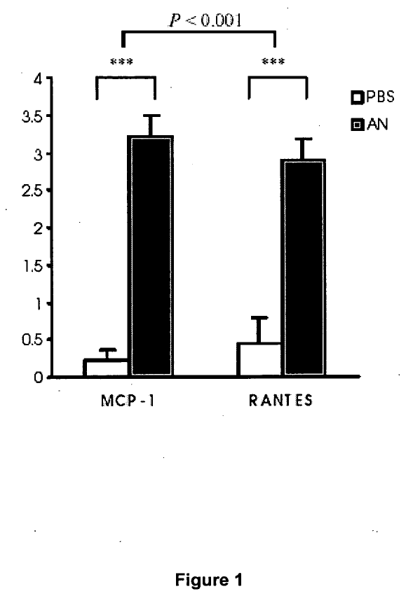

As reported previously (Rangan et al, 2000), the AN rat model was characterised by massive proteinuria immediately after adriamycin injection and progressed to renal failure. In the adriamycin rat at 4-6 weeks, severe nephrotic syndrome, glomerular sclerosis, tubular atrophy and interstitial fibrosis with massive interstitial mononuclear cell infiltration were observed. Expression of MCP-1 and RANTES mRNA in AN kidney was significantly increased compared with the controls (P<0.001) (FIG. 1).

### Example 2

### DNA Vaccination Using MCP-1 and RANTES is Protective in AN

RNA was isolated from kidneys as previously described (Walters et al, 2001) and reverse transcribed into cDNA. Oligonucleotide primers used for the MCP-1 and RANTES genes were those described as follows (Youssef et al, 1998):

| | |
|---|---|
| MCP-1: | |
| sense | |
| 5′-ATGCAGGTCTCTTGTCACGCTTCTGGGC-3′ | (SEQ ID NO:7) |
| antisense | |
| 5′-CTAGTTCTCTGTCATACTGGTCAC-3′ | (SEQ ID NO:8) |
| and | |
| RANTES: | |
| sense | |
| 5′-ATGAAGATCTCTGCAGCTGCATCC-3′ | (SEQ ID NO:9) |
| antisense | |
| 5′-CTAGCTCATCTCCAAATAGTTG-3′ | (SEQ ID NO:10) |

All forward primers were designed to include an in-frame ATG. The PCR cycle profile consisted of denaturation at 95° C. for 1 min, annealing at 55° C. for 1 min and extension at 72° C. for 1 min for 35 cycles in a DNA thermal cycler (Perkin Elmer). Amplified PCR products were cloned into the pTarget plasmid Vector (Promega, Madison, Wis.) according to the manufacturer's instructions. Plasmid DNA from colonies with an insert was sequenced to confirm the insertion of the correct gene with the ATG in-frame. Large-scale preparation of plasmid DNA was performed using the Mega prep kit (QIAGEN, Hilden, Germany).

AN rats (induced as per Example 1) were divided into five groups: group A, DV: DNA vaccination against MCP-1 and RANTES in AN (n=5); group B, DV control: DNA vaccination with empty pTarget vector as a control for vaccination in AN (n=5); group C, AN: injection with adriamycin alone (n=3); group D, PBS: injection with PBS only in normals as a control for adriamycin (n=3); group E, DV alone: DNA vaccination against MCP-1 and RANTES in normal rats (n=3).

Blood from each animal was collected weekly. At 4 weeks after adriamycin administration, rats were sacrificed and each kidney was divided into three portions: one portion was placed in formalin, one in embedding OCT compound to be snap frozen for histological examination and another portion was snap frozen and used for RT-PCR analysis. The blood and spleen from each animal were also harvested.

For DNA vaccination, animals were pre-treated with 0.75% bupivacaine (1 μl/g bodyweight; Sigma) by injection into the tibialis anterior muscle 1 wk before the first vaccination. This is known to enhance the efficiency of DNA vaccination. 200 μg of DNA was injected into the same site four times at weekly intervals. One week after the fourth DNA vaccination, rats were treated with adriamycin. Rats received a booster injection of DNA vaccine one week after adriamycin administration.

Assessment of Renal Function

Blood and 16 hour urine samples were collected in metabolic cages weekly after adriamycin administration. Urine protein concentrations were determined by colorimetric assay (Biorad, Oakland, Calif., USA). Blood and urine creatinine level 5 were analyzed as previously described (Rangan et al, 1999). Creatinine clearance was calculated as creatinine excretion divided by serum creatinine concentration.

Histology and Morphometric Analysis

Four weeks after adriamycin administration, animals were sacrificed for tissue collection. Kidneys were immersion-fixed in 10% neutral-buffered formalin and embedded in paraffin. Sections 4 μm thick were stained with periodic acid-Schiff (PAS). A computer assisted image analysis system was used to quantify glomerulosclerosis and tubular atrophy (tubular dilation and tubule cell height) in the renal cortex, as described previously (Rangan et al, 1999). Interstitial volume and interstitial infiltrates were determined by 2 blinded observers who assessed 20 high powered sections/animal. Interstitial volume was graded according to the extent of cortical involvement on a scale of 0 to 4.

Immunohistochemistry

Frozen sections were cut at 5 μm, fixed with cold acetone at 10 min and blocked with 0.3% H₂O₂ for 10 min to eliminate endogenous peroxidase and 10% goat serum for 10 min to minimize nonspecific antibody binding. Sections then were incubated with monoclonal antibodies for 60 minutes, followed by biotinylated goat anti-mouse immunoglobulin (Ig) polyclonal antibody (BD PharMingen, San Diego, Calif., USA) and avidin-biotin-horseradish peroxidase complex for 30 minutes each. The slides were incubated with 3,3-diaminobenzidine tetrahydrochloride to produce a dark brown-colored end product. The monoclonal antibodies used were W3/25 for CD4⁺, OX-8 for CD8⁺, NDS-61 for CD25⁺ cells and CD68 for macrophages (Serotec, Oxford, UK). Staining was quantitated by a blinded observer counting 20 consecutive high-power fields per animal and expressed as cells per 400× field.

Statistical Analysis

Results described herein are expressed as mean±SD. Statistical significance of difference between and among the groups was made by one way analysis of variance (ANOVA) using post hoc Fisher's PLSD analysis. A value of P<0.05 was considered significant.

Results

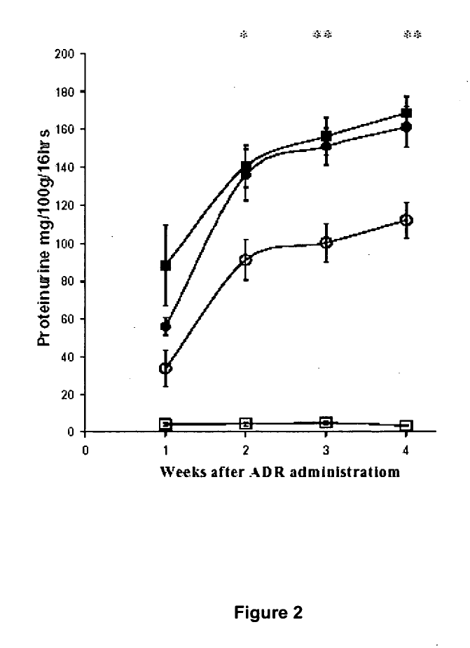

Vaccination using naked DNA encoding MCP-1 and RANTES significantly reduced proteinuria from week 1 to week 4 post adriamycin administration (FIG. 2) compared with AN and DV controls. There was no significant difference in proteinuria between AN and DV control groups. Control rats injected with only PBS did not develop proteinuria. Chemokine DNA vaccination had no effect on proteinuria in the normal rats, as there was no significant difference in proteinuria between PBS and DV alone groups at each time point.

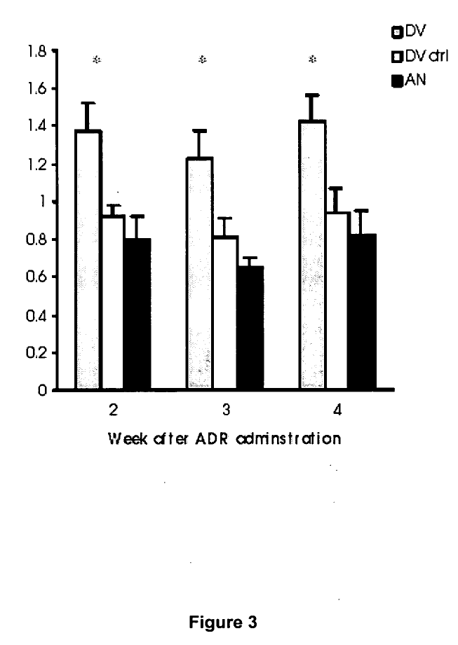

Chemokine DNA vaccination significantly ameliorated renal dysfunction in rat AN. Serum creatinine in the DV group was significantly lower than in the DV control group (47.6±15.1 vs 70.0±14.8 μmol/L, P<0.05 at week 2; 49.2±9.7 vs 70.0±10.5 μmol/L, P<0.05 at week 3) and the AN group (81.0±6.2 and 83.3±8.6 μmol/L, p<0.05 at week 2 and 3 respectively). Creatinine clearance in DV group was significantly higher than in the DV control and AN groups, p<0.05 (FIG. 3) from week 2 to week 4 post adriamycin administration. There was no significant difference in creatinine clearance between AN and DV control groups. Normal rats injected with only PBS did not develop renal failure. The DNA vaccine encoding MCP-1 and RANTES had no effect on serum creatinine and creatinine clearance in the normal rats.

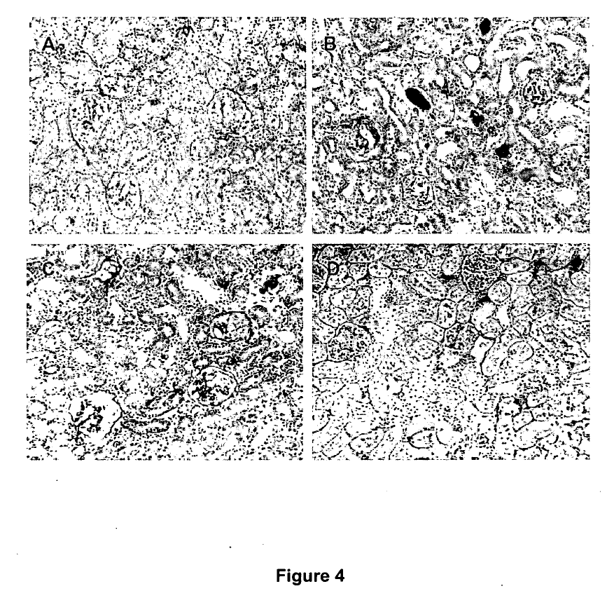

Chemokine DNA vaccination significantly alleviated the histologic manifestations of AN at week 4 (Table 1). Light microscopic examination revealed that glomeruli and tubules were only mildly damaged at week 4 in the DV group, as compared with the DV control and the AN groups (FIG. 4). Morphometric analysis showed much less glomerulosclerosis in the DV group than in the DV control and AN groups (p<0.05). Most tubules were intact in DV group rats and tubular atrophy was significantly alleviated by chemokine DNA vaccination, in comparison with rats in the AN group and DV control groups. Furthermore, vaccination with chemokine reduced interstitial infiltrates in DV group in comparison to AN group and DV control group (p<0.05). Morphological data are summarized in Table 1.

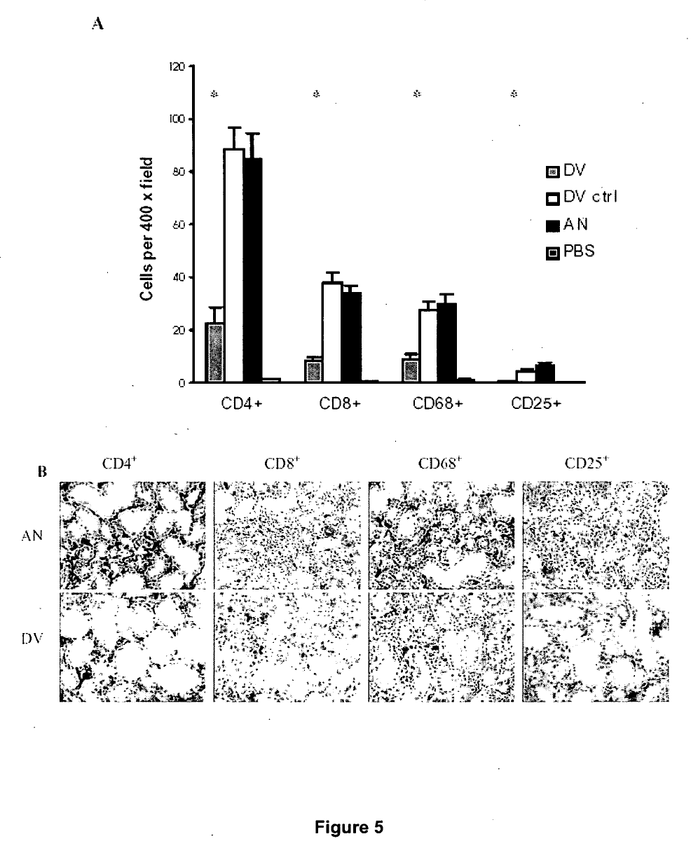

Examination of renal interstitial infiltrates in AN rats at 4 weeks demonstrated significant infiltrates of macrophages (CD68), CD8⁺ and CD4⁺ T cells, and CD25+ cells in renal interstitium as compared with PBS control rats (p<0.005), as shown in FIGS. 5A and B. Macrophages, CD8⁺ and CD4⁺ T cells, and CD25+ cells in renal interstitium in the DV group were significantly less than in the AN and DV control groups (p<0.001). There were no significant differences in staining for these cell infiltrates in the interstitium between the AN and DV control groups. These immunohistochemical data also are summarized in Table 1.

Examination of renal interstitial infiltrates in AN rats at 4 weeks demonstrated significant infiltrates of macrophages (CD68), CD8⁺ and CD4⁺ T cells, and CD25+ cells in renal interstitium as compared with PBS control rats (p<0.005), as shown in FIGS. 5A and B. Macrophages, CD8⁺ and CD4⁺ T cells, and CD25+ cells in renal interstitium in the DV group were significantly less than in the AN and DV control groups (p<0.001). There were no significant differences in staining for these cell infiltrates in the interstitium between the AN and DV control groups. These immunohistochemical data also are summarized in Table 1.

TABLE 1
Quantitation of morphology and immunohistochemistry

| | DV | DVctrl | AN | PBS | DValone |
|---|---|---|---|---|---|
| | group A | group B | group C | Group D | group E |
| Morphology | | | | | |
| Interstitial volume (0-4+) | 1.3 ± 1.2^{a} | 2.8 ± 0.8 | 3.3 ± 0.6^{c} | 0 | 0 |
| Interstitial infiltrates (0-4+) | 1.6 ± 0.9^{a} | 3.2 ± 0.8 | 3.6 ± 0.6^{c} | 0 | 0 |
| Glomerular sclerosis % | 7.6 ± 4.2^{a} | 16.4 ± 6.1 | 19.3 ± 1.2^{c} | 0 | 0 |
| Tubular diameter μm | 34.6 ± 1.6^{a} | 31.5 ± 2.1 | 31.5 ± 0.6^{c} | 41.6 ± 0.9 | 42.3 ± 0.8 |
| Tubular cell height μm | 10.9 ± 0.3^{a} | 9.9 ± 0.9 | 10.3 ± 0.2^{c} | 12.8 ± 0.3 | 12.5 ± 0.2 |
| Immunohistochemistry | | | | | |
| (cells/400 × field) | | | | | |
| CD4⁺ cells | 22.6 ± 13.3^{b} | 88.8 ± 18.2 | 84.9 ± 16.8^{d} | 1.1 ± 1.4 | 1.4 ± 1.2 |
| CD8⁺ cells | 8.3 ± 3.6^{b} | 37.7 ± 9.2 | 33.9 ± 5.1^{d} | 0.8 ± 0.2 | 0.4 ± 0.3 |
| Macrophages (CD68′) | 8.9 ± 3.9^{b} | 27.7 ± 7.3 | 29.6 ± 6.8^{d} | 1.8 ± 0.5 | 0.6 ± 0.4 |
| CD25+ cells | 0.42 ± 0.3^{b} | 4.5 ± 0.6 | 6.4 ± 2.2^{d} | 0.2 ± 0.1 | 0.1 ± 0.1 |

Value are expressed as mean ± SD.
^{a}p < 0.05 compared with group 2 and group 3.
^{b}P < 0.001 compared with group 2 and group 3.
^{c}p < 0.001 compared with group 4 and group 5.
^{d}p < 0.005 compared with group 4 and group 5.

### Example 3

### Anti-Chemokine Antibodies Produced by MCP-1 and RANTES DNA Vaccination In Vivo

To test whether vaccination using DNA encoding MCP-1 and RANTES induced the production of autoantibodies against gene products of each vaccinated DNA, serum samples of all animals from week 2 to week 4 were analyzed for specific autoantibodies against MCP-1 and RANTES by ELISA.

Anti-MCP-1 or anti-RANTES antibody titres (total Ig) were determined by a direct ELISA assay as described (Youssef et al, 1998; Wu et al, 2001). Briefly, 96 wells of an Immulon 1 ELISA microtiter plate (Dynatch Laboratories, Alexandria, Va.) were coated with each recombinant rat chemokine MCP-1 and RANTES (Chemicon) at a concentration of 25 ng/well in 100 μl of coating buffer and reacted sequentially with test rat sera, alkaline phosphatase conjugated sheep anti-rat Ig Fab fragments (Boehringer Mannheim, GmbH, Mannheim, Germany) and substrate solution (0.5% p-nitrophenyl phosphate (Sigma) in carbonate buffer, pH 9.6. Absorbance was read at 405 nm on an ELISA reader (Dynatech Laboratories). Triplicate sample ODs were read at 405 nm, corrected for a control sample of known strongly positive serum OD (i.e., sample OD/control positive serum OD×100). The anti-MCP-1 or anti-RANTES antibody titer of the control serum was 1:200.

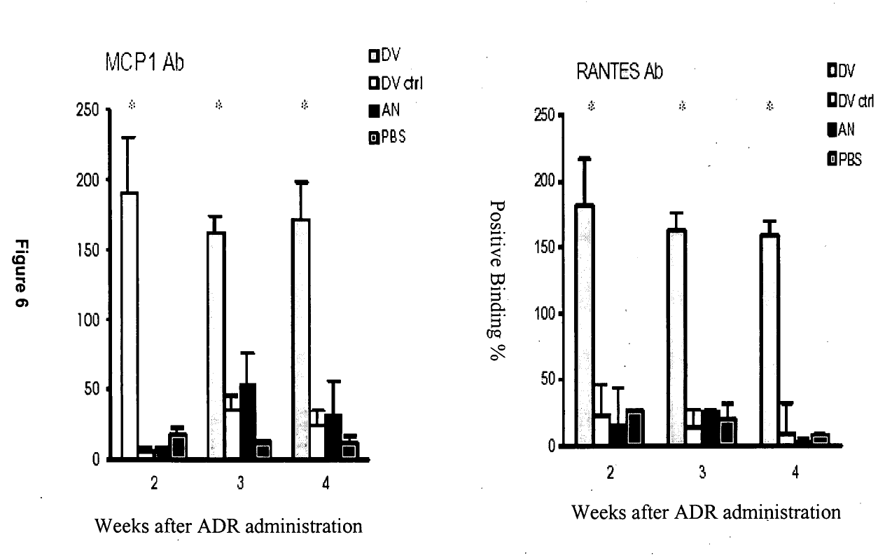

Rats vaccinated with chemokine DNA had significantly higher levels of MCP-1 and RANTES autoantibody titers than rats in the AN, DV control group and PBS groups (P<0.005), as shown in FIG. 6.

### Example 4

### Modification of MCP-1 to Generate a Hybrid Protein

The rat MCP-1 gene was modified by replacing a surface loop region +109 to +138 (37 to 46 aa) by P30 tetanus toxoid helper epitope sequence TTCACCAACTTCACCGTCAGCTTCTGGCTGCGCGTGCCCAAGGTCAGCGCCAGCCAC CTGGAG (SEQ ID NO:11) (encoding FNNFTVSFWLRVPKVSASHLE; SEQ ID NO:12) using primer extension PCR.

Wild-type rat MCP-1 cDNA was amplified by RT-PCR from RNA extracts from kidney of AN rats. The primers used were as follows:

CCACTATGCAGGTCTCTGTC (SEQ ID NO:13) MCP-1 5′ primer designed for amplification of the first overlap fragment of modified MCP-1;

ATCACATTCCAAATCACACTAG (SEQ ID NO:14) MCP-1 3′ primer (reverse) designed for amplification of the second overlap fragment of modified MCP-1;

GACCTTGGGCACGCGCAGCCAGAAGCTGACGGTGAAGTTGGTGAAgtagcagca ggtgagtgg (SEQ ID NO:15) overlap primer 1 (reverse) from overlap region of P30 to adjacent MCP-1 sequence (lower case) (sequence on antisense chain); and

TGGCTGCGCGTGCCCAAGGTCAGCGCCAGCCACCTGGAGatgagtcggctggagaact a (SEQ ID NO:16) overlap primer 2 (forward) from overlap region of P30 to adjacent MCP-1 sequence (lower case).

MCP-1 5′ primer and overlap primer 1 were used to amplify a first fragment of modified MCP-1 gene, while overlap primer 2 and MCP-1 3′ primer were used to amplify a second fragment of modified MCP-1. The first fragment was annealed to the second fragment at the overlap region. This was then further extended and amplified with 5′ and 3′ MCP-1 primers. The modified MCP-1 PCR product was cloned into pTarget vector (Promega, Madison, Wis.) to make the modified MCP-1 (ΔMCP-1) DNA vaccine. Plasmid DNA was prepared in large scale using Qiagen Plasmid Mega Kit.

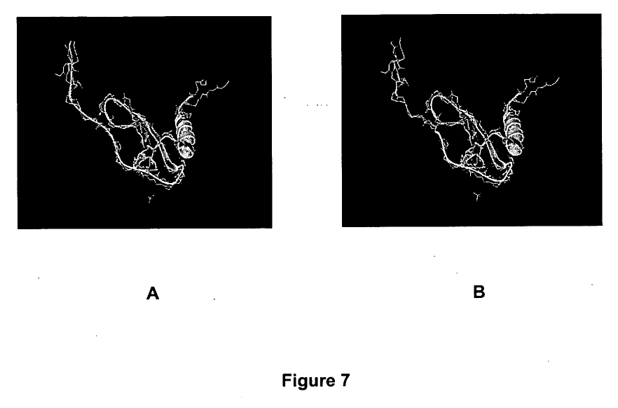

The modification was verified by sequencing the clonal product of plasmid constructs. Of five clones screened, clone 3.2 gave a sequence exactly as designed. Clone 3.2 was prepared further in large scale. The three dimensional (3D) structure of modified rat MCP-1 protein was predicted and compared with wild-type using SWISS-MODEL online (http://swissmodel.expasy.org/). 3D structure prediction revealed that after modification, the natural folding of wild-type MCP-1 protein was maintained in the modified hybrid MCP-1 and that the P30 peptide was located at surface loop region (see FIG. 7; the portion of the polypeptide bounded by the arrows).

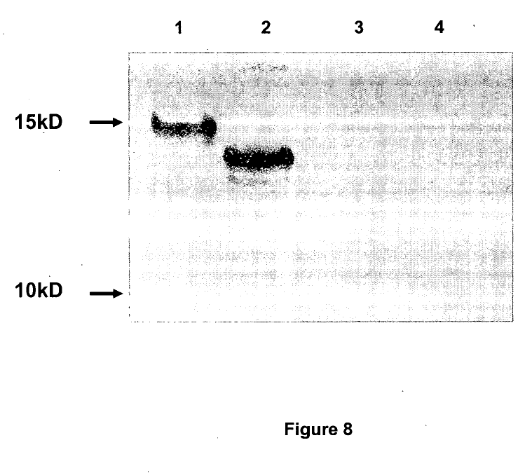

To confirm that the modified MCP-1 DNA vaccine is capable of expressing the hybrid protein in a mammalian expression vector, HeLa cells, which do not express MCP-1, were transiently transfected with plasmid DNA of pTarget vector, pTarget/MCP-1 and pTarget/ΔMCP-1 using lipofactamine™ 2000 (Invitrogen, Carlsbad, Calif., USA). Cell lysates were subjected to immunoblotting with rabbit anti-rat MCP-1 (Chemicon) followed by goat anti-rabbit Ig:HRP, and detected with a chemilucent ECL detection system (Chemicon). Modified MCP-1 DNA vaccine expressed a hybrid protein with a slightly higher molecular weight (15 kDa) than the wild type (14 kDa), and could be recognized by anti rat MCP-1 Ab (FIG. 8).

### Example 5

### Vaccination Using Modified MCP-1 Construct

The efficacy of a vaccine comprising a polynucleotide encoding wild-type rat MCP-1 in treatment of AN was compared to that of a vaccine comprising a polynucleotide encoding a modified MCP-1 in which the surface loop region at bases +109 to +138 (37 to 46 aa) was replaced by a P30 tetanus toxoid helper epitope sequence (see Example 4).

Male Wistar rats approximately 4-5 weeks old, weighing at 90-110 g were purchased from the Australian Research Council and maintained under clean conditions in the Department of Animal Care at Westmead Hospital. Experiments were carried out in accordance with protocols approved by Animal Ethics Committee of Western Sydney Area Health Service. Adriamycin nephropathy (AN) was induced by a single tail vein injection of Adriamycin (ADR 5 mg/Kg; David Bull Labs, Victoria, Australia).

Animals were divided randomly into five groups: normal control: (n=4), Adriamycin control (n=6), vector control (n=6), unmodified MCP-1 vaccine (n=6) and modified MCP-1 vaccined (n=7). Rats were pretreated with 0.75% bupivacaine (1 μl/g bodyweight; Sigma) by intramuscular injection into tibialis anterior muscle one week prior to plasmid DNA vaccination. 300 μg plasmid DNA prepared in large scale was injected weekly on four occasions into the same site as bupivacaine. One week after the fourth DNA vaccination, AN was induced by tail vein injection of adriamycin.

Renal function was measured as described in Example 2.

For histopathology, four weeks after adriamycin administration, animals were sacrificed. A coronal slice of kidney from each animal was fixed in 10% neutral buffered formalin for 24 hours and then dehydrated in graded alcohols and embedded in paraffin. Tissues were cut at 5 μM and stained with periodic acid-shiff (PAS) and hematoxylin. Glomerulosclerosis, tubular atrophy and interstitial expansion and infiltrates were semiquantitated using a score of severity from 0 to +3. The slides were read by an observer blinded to group identifications, and 20×200 fields assessed for each section. The results are expressed as mean±SE.

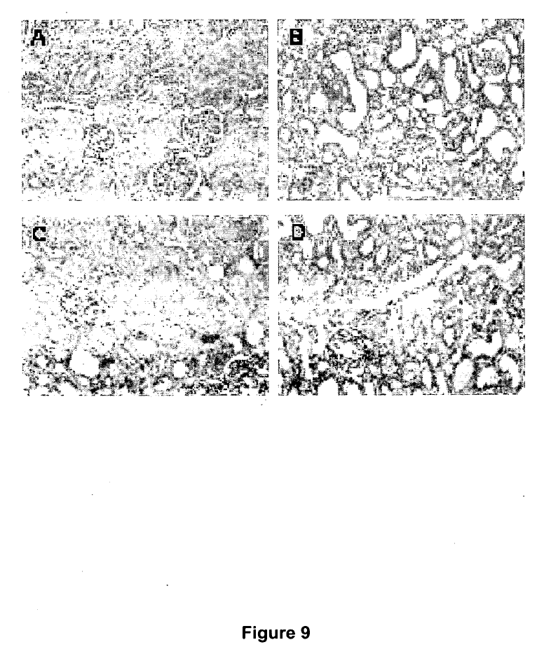

Rats vaccinated with modified MCP-1 DNA vaccine developed much less severe renal structural injury at week 4 of AN. Glomerular and tubular damage was only mild in rats vaccinated with modified MCP-1 DNA, in comparison to very severe glomerular and tubular injury in rats from adriamycin control, vector control and unmodified MCP-1 vaccine groups (FIG. 9). Semiquantitative morphometrics (Table 2) showed less glomerular sclerosis and interstitial expansion in rats vaccinated with modified MCP-1 compared to that of rats vaccinated with vector control or unmodified MCP-1. No significant difference in tubular atrophy was found among the groups.

TABLE 2
Morphological changes in AN rats following vaccination with
wild-type or modified MCP-1 encoding polynucleotides

| | | Vector | MCP-1 | |
|---|---|---|---|---|
| | | control | vaccine | mMCP-1 vaccine |
| | | n = 6 | n = 6 | n = 7 |
| Overall morphology | (0-3+) | 3.0 ± 0 | 2.7 ± 0.6 | 1.8 ± 0.9* |
| Glomerular sclerosis | (0-3+) | 2.5 ± 0.5 | 2.0 ± 0 | 0.8 ± 0.9** |
| Tubular atrophy | (0-3+) | 2.7 ± 0.6 | 2.5 ± 0.5 | 2.0 ± 0.4 |
| Interstitial infiltrates | (0-3+) | 3.0 ± 0 | 2.5 ± 0.5 | 1.4 ± 0.5** |

*p < 0.05 compared with vector control group.
**p < 0.05 compared with vector control and unmodified MCP-1 vaccine groups.

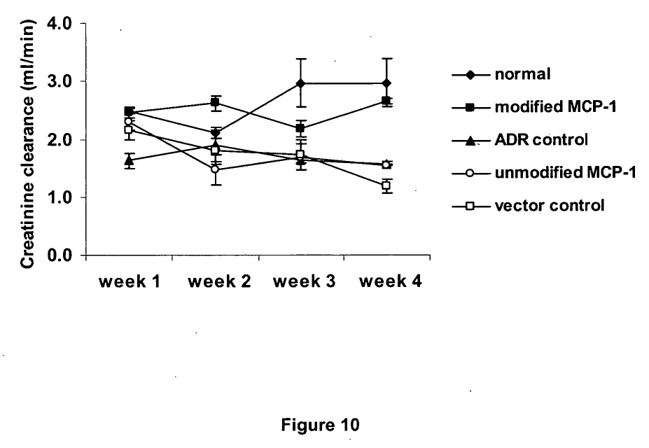

Renal function also was protected significantly by vaccination with modified MCP-1 vaccine. Creatinine clearance was significantly higher in rats vaccinated with modified MCP-1 than that of ADR control, vector control and unmodified MCP-1 vaccine groups at week 4 (2.65 ±0.04 vs 1.57±0.04, 1.19±0.12 and 1.54±0.01 ml/min, P<0.05) (FIG. 10) and was no different from normal control (2.97±0.4). However, 16 hour urine protein secretion in rats vaccinated with modified MCP-1 was not significantly different from that of ADR control, vector control and unmodified MCP-1 vaccine groups.

### Example 6

### Production of Autoantibodies Against MCP-1 and MIP-1α by Vaccination with Modified MCP-1 DNA

A direct ELISA assay as described (Youssef et al., 1998) was used to determine the anti-MCP-1 or anti-MIP1α Ab titer in sera from DNA vaccinated rats. Recombinant rat MCP-1 and MIP1α were coated onto 96-well ELISA plates (Titertek®) at a concentration of 50 ng/well in 100 μl coating buffer. Rat sera, in serial dilution from 2⁶ to 2²⁰ or in one dilution (1:100) were added to coated ELISA plates. Goat anti-rat IgG alkaline phosphatase-conjugated Ab (Sigma) and p-Nitrophenyl phosphate (Sigma) as substrate were used sequentially for the Ab titer analysis. Absorbance at 405 nm was read by an ELISA reader (Biorad). The results are expressed as Ab titer dilution±SE or absorbance at 405 nm±SE when one dilution assay was used.

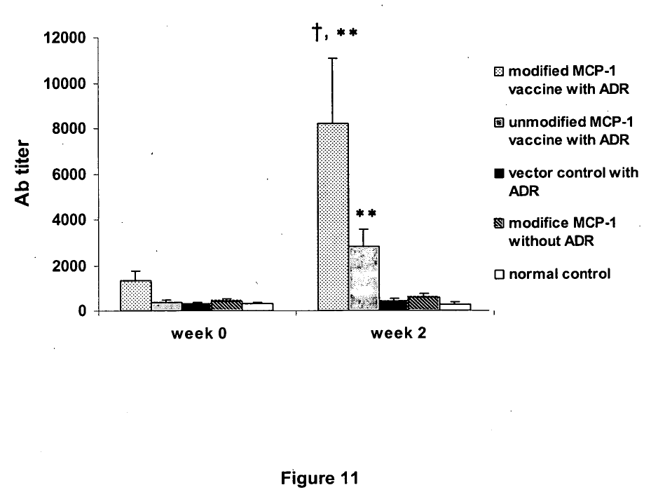

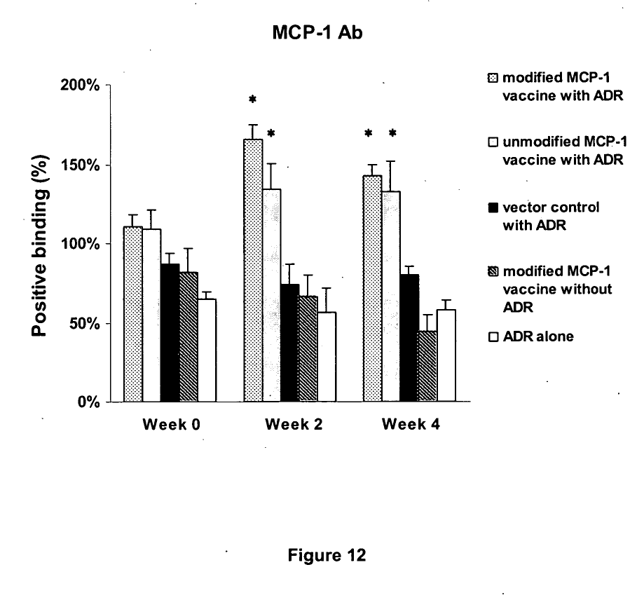

Self-specific Ab against MCP-1 was produced in sera of rats after vaccination with modified MCP-1 DNA as well as unmodified MCP-1 DNA. However, the Ab titer in the sera of rats vaccinated with modified MCP-1 was significantly higher than that of rats vaccinated with unmodified MCP-1 at week 2 of AN (8192±2896 vs 2816±768), the time of peak interstitial macrophage infiltration (FIG. 11). High level autoantibody was sustained through out the disease course until week 4 when animals were sacrificed (FIG. 12). Ab titer of normal rats vaccinated with modified MCP-1 (576±161, P<0.001) was significantly lower than that of AN rats vaccinated with modified or unmodified MCP-1, but not different from that of AN rats vaccinated with vector control and normal control (448±64 and 288±80 respectively, P=0.9).

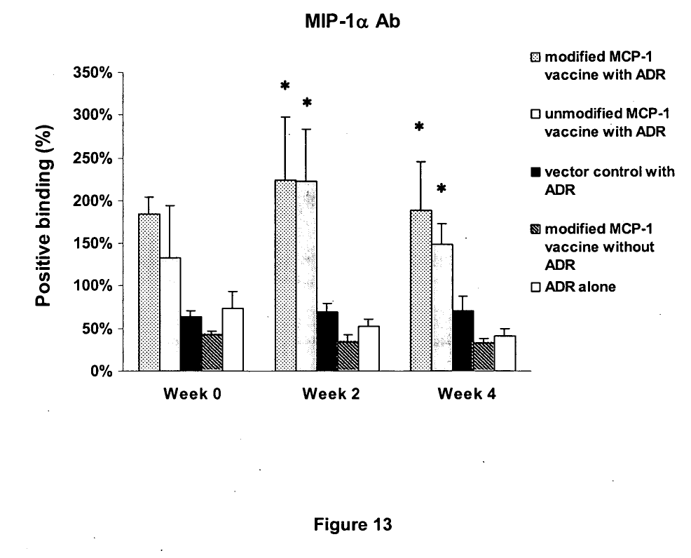

As cross reactivity of Ab against CC-chemokine DNA vaccine towards homologous CC-chemokines has been reported elsewhere (Youssef et al., 2000), anti-sera from rats vaccinated with modified MCP-1 was tested also for Ab to macrophage inflammatory protein-1α (MIP-1α) and RANTES by ELISA using one dilution of the antisera (1:100). Anti-sera binding to rMIP-1α was significantly higher in rats vaccinated with modified MCP-1 as well as unmodified MCP-1 compared to that of the vector control group (FIG. 13), but not to rRANTES (data not shown).

### Example 7

### Increased IFN-γ Producing T Cells from Draining Lymph Nodes Following Modified MCP-1 DNA Vaccination

To assess the cellular response of modified MCP-1 DNA vaccine in comparison to unmodified vaccine and vector control, the numbers of IFN-γ producing T cells from draining lymph nodes of vaccinated normal rats were assessed by enzyme-linked immunospot (ELISpot) assay.

ELISpot assay was performed according BD™ ELISpot protocol. The 96-well multiscreen plates (Millipore) were coated with capture Ab, mouse anti-rat IFN-γ (Biosource; 25 ng/well). Lymphocytes from popliteal draining lymph nodes (DLN) of DNA vaccinated rats were isolated and added in triplicate to the ELISpot plate at 3×10⁵ cells/well in RPMI 1640 with 5% FCS, then stimulated with rMCP-1 at concentrations of 0, 25, 50 and 10 ng/well by incubated at 37° C., 5% CO₂ for 24 hours. The plates were detected with detection Ab, biotin conjugated mouse anti-rat IFN-γ (Biosource; 25 ng/well) at 4° C. over night, incubated with streptavidin alkaline phosphatase (Becton Dickinson) at room temperature for 2 hours, and then developed with BCIP/NBT kit (Biorad). Spots were counted under a dissecting microscope. The results were expressed as number of spot-forming cells/10⁶ DLN cells.

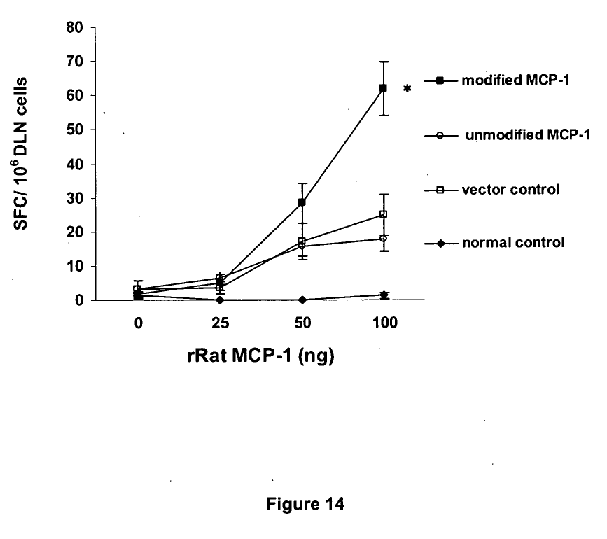

Rats vaccinated with modified MCP-1 DNA had more T cells from draining lymph nodes that produced IFN-γ when stimulated with 10 ng/ml rMCP-1 in vitro than rats vaccinated with unmodified MCP-1 or vector control rats (62.0±7.8 vs 17.7±3.5 and 25.0±6.1, P<0.001). The number of IFN-γ producing T cells of rats vaccinated with unmodified MCP-1 vaccine was not different to that of vector control (FIG. 14).

### Example 8

### Protection of AN is Associated with Reduced Interstitial Macrophage Infiltration

Immunohistochemical staining of interstitial inflammatory cells (macrophage, CD4+ and CD8+ T cells) was carried out. Frozen sections were cut at 6 μM from cortical slices of kidneys embedded in OCT compound (Tissue-Tek®, Sakura Finetek, Torrance, Calif., USA), fixed with acetone at 4° C. for 10 mins and immersed in 0.3% H₂O₂ for 10 mins to eliminate endogenous peroxidase and blocked with Background Buster (AXELL, Westbury, N.Y., USA) at 10 mins each to minimize background and nonspecific Ab binding. Sections were incubated individually with mAb against macrophages (CD68+), CD8+ and CD4+ T cells (Serotec) for 90 mins, followed by goat anti-mouse IgG: HRP conjugated secondary Ab (Serotec) and visualized by addition of freshly prepared 3,3-diaminobenzidine tetrahydrochloride. The number of macrophages, CD8+ and CD4+ T cells was quantitated in 10 non-overlapping cortical fields (×400), expressed as number of cells per×400 field per animal.

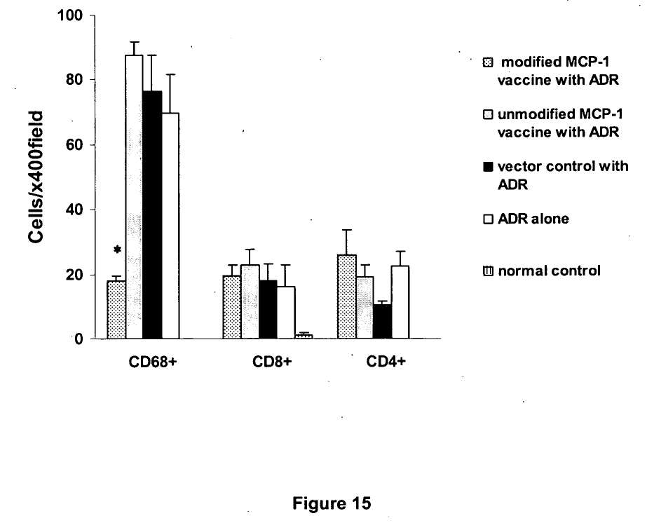

Immunohistochemical staining revealed that modified MCP-1 DNA vaccination significantly reduced the number of interstitial macrophages compared to unmodified MCP-1 DNA vaccine, vector control and AN control (18.0±1.4 vs 87.5±4.3, 76.3±11.4 and 69.5±12.3 cells/400× field, P<0.001) (FIG. 15); there was no difference amongst unmodified MCP-1, vector control and AN control groups. There was no significant difference in numbers of interstitial CD8+ and CD4+ T cells amongst the groups.

### Example 9

### Antisera Neutralization of Chemoattractive Effect of MCP-1 Towards Monocytes In Vitro

To explore the mechanism whereby modified MCP-1 DNA vaccination reduced interstitial macrophages, the effect of anti-sera was tested in vitro for its blocking effect against MCP-1 induced monocyte chemotaxis.

The chemotaxis assay was performed according to Youssefet al. (1998) with modification. Rat recombinant MCP-1 (rMCP-1) (Chemicon, 10 ng/ml) in RPMI 1640 was added to lower well of 6-well plate with or without preincubation with the sera of DNA vaccinated rats. Monocytes prepared by Lymphoprep™ (Axia-Shield PoC AS, Oslo, Norway) from rat splenocytes were seeded onto upper wells (insert with polycarbonate membrane of 8 μM pore size) (Nunc) at 4×10⁶/ml in RPMI 1640 enriched with 1% BSA, and incubated at 37° C., 5% CO₂ for 2 hours. Cells that migrated to lower wells were harvested by trypsinization, and the total number of migrated cells in each well was counted by hemocytometer. The results are expressed as total number of migrated cells after subtracting the number of cells in control wells without rMCP-1.

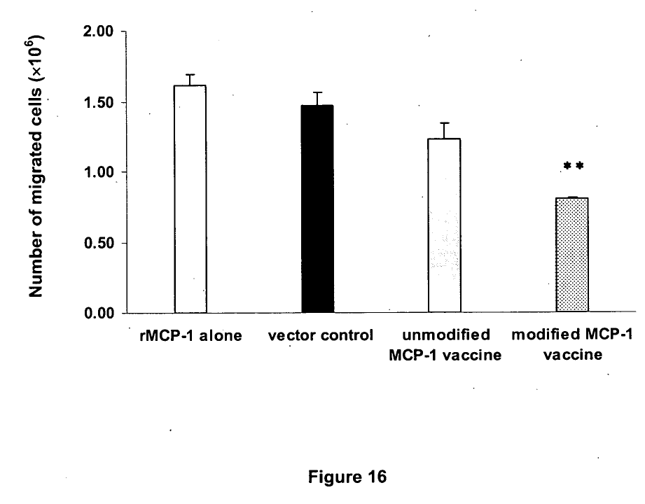

The total number of migrated monocytes was significantly lower under antisera of rats vaccinated with modified MCP-1 than under antisera from rats vaccinated with unmodified MCP-1 DNA or vector control, or rMCP-1 only control wells (80±1 vs 123±10, 147±9 and 161±8×10⁴ cells, P<0.001) (FIG. 16).

### Example 10

### Compositions for Treatment

In accordance with the best mode of performing the invention provided herein, specific preferred compositions are outlined below. The following are to be construed as merely illustrative examples of compositions and not as a limitation of the scope of the present invention in any way.

### Example 10(A)

### Composition for Parenteral Administration

A composition for intramuscular injection could be prepared to contain 1 mL sterile buffered water, and 1 mg of a suitable agent or compound.

Similarly, a composition for intravenous infusion may comprise 250 ml of sterile Ringer's solution, and 5 mg of a suitable agent or compound.

### Example 10(B)

### Injectable Parenteral Administration

A composition suitable for administration by injection may be prepared by mixing 1% by weight of a suitable agent or compound in 10% by volume propylene glycol and water. The solution is sterilised by filtration.

### Example 10(C)

### Capsule Composition

A composition of a suitable agent or compound in the form of a capsule may be prepared by filling a standard two-piece hard gelatin capsule with 50 mg of the agent or compound, in powdered form, 100 mg of lactose, 35 mg of talc and 10 mg of magnesium stearate.

### REFERENCES

Anders H J, Vielhauer V, Schlondorff D: Chemokines and chemokine receptors are involved in the resolution or progression of renal disease. Kidney Int. 63:4011-415, 2003.
Dalum I, Jensen M R, Hindersson P, Elsner H I, Mouritsen S: Breaking of B cell tolerance toward a highly conserved self protein. J Immunol 157:4796-4804, 1996.
Fujinaka H, Yamamoto T, Takeya M, Feng L, Kawasaki K, Yaoita E, Kondo D, Wilson C B, Uchiyama M, Kihara I: Suppression of anti-glomerular basement membrane nephritis by administration of anti-monocyte chemoattractant protein-1 antibody in WKY rats. J Am Soc Nephrol 8:1174-1178, 1997.
Hertz M, Mahalingam S, Dalum I, Klysner S, Mattes J, Neisig A, Mouritsen S, Foster P S, Gautam A: Active vaccination against IL-5 bypasses immunological tolerance and ameliorates experimental asthma. J Immunol 167:3792-3799, 2001.

Lloyd C M, Minto A W, Dorf M E, Proudfoot A, Wells T N, Salant D J, Gutierrez-Ramos J C: RANTES and monocyte chemoattractant protein-1 (MCP-1) play an important role in the inflammatory phase of crescentic nephritis, but only MCP-1 is involved in crescent formation and interstitial fibrosis. J. Exp. Med. 185:1371-1380, 1997.
Rangan G K, Wang Y, Tay Y C, Harris D C: Inhibition of nuclear factor-kappaB activation reduces cortical tubulointerstitial injury in proteinuric rats. Kidney Int. 56:118-134, 1999.
Rangan G K, Wang Y, Tay Y C, Harris D C: Cytokine gene expression in Adriamycin nephropathy: effects of antioxidant nuclear factor kappaB inhibitors in established disease. Nephron 86:482-490, 2000.
Rangan G K, Wang Y, Harris D C: Induction of proteinuric chronic glomerular disease in the rat (Rattus norvegicus) by intracardiac injection of doxorubicin hydrochloride. Contemp. Top. Lab Anim Sci. 40:44-49, 2001.
Segerer S: The role of chemokines and chemokine receptors in progressive renal diseases. Am. J. Kidney Dis. 41:S15-S18, 2003.
Segerer S, Nelson P J, Schlondorff D: Chemokines, chemokine receptors, and renal disease: from basic science to pathophysiologic and therapeutic studies. J. Am. Soc. Nephrol. 11:152-176, 2000.
Walters G, Wu H, Knight J F: Glomerular T cells in Heymann nephritis. Clin Exp Immunol 126(2):319-325, 2001.
Wang Y, Chen J, Chen L, Tay Y C, Rangan G K, Harris D C: Induction of monocyte chemoattractant protein-1 in proximal tubule cells by urinary protein. J. Am. Soc. Nephrol. 8:1537-1545, 1997.
Wang Y, Wang Y, Tay Y, Harris D: Progressive adriamycin nephropathy in mice: sequence of histologic and immunohistochemical events. Kidney Int 58:1797-1804, 2000.
Wu X, Dolecki G J, Sherry B, Zagorski J, Lefkowith J B: Chemokines are expressed in a myeloid cell-dependent fashion and mediate distinct functions in immune complex glomerulonephritis in rat. J Immunol 158:3917-3924, 1997.
Wu H, Zhang G Y, Knight J F: T cell receptor BV gene usage in interstitial cellular infiltrates in active Heymann nephritis. Nephrol Dial Transplant 16:1374-1381, 2001.
Youssef S, Wildbaum G, Maor G, Lanir N, Gour-Lavie A, Grabie N, Karin N: Long-lasting protective immunity to experimental autoimmune encephalomyelitis following vaccination with naked DNA encoding C-C chemokines. J. Immunol. 161:3870-3879, 1998.
Youssef S, Maor G, Wildbaum G, Grabie N, Gour-Lavie A, Karin N: C-C chemokine-encoding DNA vaccines enhance breakdown of tolerance to their gene products and treat ongoing adjuvant arthritis. J Clin Invest 106:361-371, 2000.

What is claimed is:

1. A method for the treatment or prevention of renal disease in a subject, the method comprising administering to the subject an effective amount of a polynucleotide encoding MCP-1 operably linked to a promoter.

2. The method of claim 1 wherein the polynucleotide encoding MCP-1 has the nucleotide sequence set forth in SEQ ID NO:1.

3. The method of claim 1 wherein the method further comprises administering to the subject an effective amount of a polynucleotide encoding RANTES operably linked to a promoter.

4. The method of claim 3 wherein the polynucleotide encoding RANTES has the nucleotide sequence set forth in SEQ ID NO:3.

5. The method of claim 1 wherein the polynucleotide encodes a hybrid MCP-1 polypeptide.

6. The method of claim 5 wherein in the hybrid polypeptide a surface loop region of MCP-1 is replaced with a corresponding region of a second protein.

7. The method of claim 6 wherein in the hybrid polypeptide a surface loop region of MCP-1 is replaced with the P30 tetanus toxoid T helper epitope.

8. The method of claim 7 wherein the polynucleotide encoding hybrid MCP-1 has the nucleotide sequence set forth in SEQ ID NO:5.

9. The method of claim 1 wherein the renal disease is a chronic proteinuric renal disease.

10. The method of claim 1 wherein the renal disease is selected from the group consisting of: focal glomerulosclerosis, glomerulonephritis, diabetic renal disease, hypertensive renal disease, renal failure, and end-stage renal disease.

11. The method of claim 3 wherein the polynucleotide encoding MCP-1 and the polynucleotide encoding RANTES are located in a single nucleic acid construct.

12. The method of claim 1 wherein administration of the polynucleotide induces an immune response in the subject.

13. A composition for the treatment or prevention of renal disease comprising a polynucleotide encoding MCP-1 operably linked to a promoter.

14. The composition of claim 13 wherein the polynucleotide encoding MCP-1 has the nucleotide sequence set forth in SEQ ID NO:1.

15. The composition of claim 13 further comprising a polynucleotide encoding RANTES operably linked to a promoter.

16. The composition of claim 15 wherein the polynucleotide encoding RANTES has the nucleotide sequence set forth in SEQ ID NO:3.

17. The composition of claim 13 wherein the polynucleotide encodes a hybrid MCP-1 polypeptide.

18. The composition of claim 17 wherein in the hybrid polypeptide a surface loop region of MCP-1 is replaced with a corresponding region of a second protein.

19. The composition of claim 18 wherein in the hybrid polypeptide a surface loop region of MCP-1 is replaced with the P30 tetanus toxoid T helper epitope.

20. The composition of claim 19 wherein the polynucleotide encoding hybrid MCP-1 has the nucleotide sequence set forth in SEQ ID NO:5.

21. The composition of claim 13 wherein the composition is a DNA vaccine.

22. A method for inducing protective immunity against renal disease in a subject, the method comprising administering to the subject an effective amount of a polynucleotide encoding MCP-1 operably linked to a promoter.

23. The method of claim 22 wherein the method further comprises administering to the subject an effective amount of a polynucleotide encoding RANTES operably linked to a promoter.

24. The method of claim 23 wherein the polynucleotide encoding MCP-1 has the nucleotide sequence set forth in SEQ ID NO:1 or SEQ ID NO:5 and the polynucleotide encoding RANTES has the nucleotide sequence set forth in SEQ ID NO:3.

25. The method of claim 22 wherein the polynucleotide encodes a hybrid MCP-1 polypeptide.

26. The method of claim 25 wherein in the hybrid polypeptide a surface loop region of MCP-1 is replaced with the P30 tetanus toxoid T helper epitope.

27. An immunological composition comprising a polynucleotide encoding MCP-1 operably linked to a promoter, wherein administration of the composition to a subject induces an immune response in the subject.

28. The composition of claim 27 further comprising a polynucleotide encoding RANTES operably linked to a promoter.

29. The composition of claim 28 wherein the polynucleotide encoding MCP-1 has the nucleotide sequence set forth in SEQ ID NO:1 or SEQ ID NO:5 and the polynucleotide encoding RANTES has the nucleotide sequence set forth in SEQ ID NO:3.

30. The composition of claim 27 wherein the polynucleotide encodes a hybrid MCP-1 polypeptide.

31. The composition of claim 30 wherein in the hybrid polypeptide a surface loop region of MCP-1 is replaced with the P30 tetanus toxoid T helper epitope.

32. The composition of claim 27 wherein the composition is a DNA vaccine.


SEQUENCE LISTING (16 sequences)

SEQ ID NO: 1 - DNA, 300 bp, Homo sapiens
atgaaagtctctgccgcccttctgtgcctgctgctcatagcagccaccttcattccccaagggctcgctcagccagatgcaatcaatgccccagtcacctgctgttataacttcaccaataggaagatctcagtgcagaggctcgcgagctatagaagaatcaccagcagcaagtgtcccaaagaagctgtgatcttcaagaccattgtggccaaggagatctgtgctgaccccaagcagaagtgggttcaggattccatggaccacctggacaagcaaacccaaactccgaagacttga

SEQ ID NO: 2 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

SEQ ID NO: 3 - DNA, 276 bp, Homo sapiens
atgaaggtctccgcggcacgcctcgctgtcatcctcattgctactgccctctgcgctcctgcatctgcctccccatattcctcggacaccacaccctgctgctttgcctacattgcccgcccactgccccgtgcccacatcaaggagtatttctacaccagtggcaagtgctccaacccagcagtcgtctttgtcacccgaaagaaccgccaagtgtgtgccaacccagagaagaaatgggttcgggagtacatcaactctttggagatgagctag

SEQ ID NO: 4 - protein, 91 aa, Homo sapiens
MKVSAARLAVILIATALCAPASASPYSSDTTPCCFAYIARPLPRAHIKEYFYTSGKCSNPAVVFVTRKNRQVCANPEKKWVREYINSLEMS

SEQ ID NO: 5 - DNA, 483 bp, Artificial
  Synthetic sequence
atgcaggtctctgtcacgcttctgggcctgttgttcacagttgctgcctgtagcatccacgtgctgtctcagccagatgcagttaatgccccactcacctgctgctacttcaccaacttcaccgtcagcttttggctgcgcgtgcccaaggtcagcgccagccacctggagatgagtcggctggagaactacaagagaatcaccagcagcaggtgtcccaaagaagctgtagtatttgtcaccaagctcaagagagagatctgtgctgaccccaataaggaatgggtccagaagtacattagaaaactggaccagaaccaagtgagatcagaaactacagtcttctataaaattgcatcaaccctaaggacttcagcacctttgaatgtgaacttgacccataaatctgaagctaatgcatccactctcttttccacaaccacctcaagcacttctgtagaagtgaccagtatgacagagaac

SEQ ID NO: 6 - protein, 161 aa, Artificial
  Synthetic sequence
MQVSVTLLGLLFTVAACSIHVLSQPDAVNAPLTCCYFTNFTVSFWLRVPKVSASHLEMSRLENYKRITSSRCPKEAVVFVTKLKREICADPNKEWVQKYIRKLDQNQVRSETTVFYKIASTLRTSAPLNVNLTHKSEANASTLFSTTTSSTSVEVTSMTEN

SEQ ID NO: 7 - DNA, 28 bp, Artificial
  Synthetic sequence
atgcaggtctcttgtcacgcttctgggc

SEQ ID NO: 8 - DNA, 24 bp, Artificial
  Synthetic sequence
ctagttctctgtcatactggtcac

SEQ ID NO: 9 - DNA, 24 bp, Artificial
  Synthetic sequence
atgaagatctctgcagctgcatcc

SEQ ID NO: 10 - DNA, 22 bp, Artificial
  Synthetic sequence
ctagctcatctccaaatagttg

SEQ ID NO: 11 - DNA, 63 bp, Artificial
  Synthetic sequence
ttcaccaacttcaccgtcagcttctggctgcgcgtgcccaaggtcagcgccagccacctggag

SEQ ID NO: 12 - protein, 21 aa, Artificial
  Synthetic sequence
FNNFTVSFWLRVPKVSASHLE

SEQ ID NO: 13 - DNA, 20 bp, Artificial
  Synthetic sequence
ccactatgcaggtctctgtc

SEQ ID NO: 14 - DNA, 22 bp, Artificial
  Synthetic sequence
atcacattccaaatcacactag

SEQ ID NO: 15 - DNA, 63 bp, Artificial
  Synthetic sequence
gaccttgggcacgcgcagccagaagctgacggtgaagttggtgaagtagcagcaggtgagtgg

SEQ ID NO: 16 - DNA, 59 bp, Artificial
  Synthetic sequence
tggctgcgcgtgcccaaggtcagcgccagccacctggagatgagtcggctggagaacta